(19) United States
(12) Patent Application Publication
(10) Pub. No.: US 2026/0363728 A1
(43) Pub. Date: Nov. 19, 2026

(54) SURGICAL KNIVES AND RELATED METHODS OF USING THE SAME

(71) Applicant: WAKE FOREST UNIVERSITY HEALTH SCIENCES, Winston-Salem, NC (US)

(72) Inventors: Christopher Kyle CRAIG, Winston-Salem, NC (US); Joseph Daniel CARTER, Winston-Salem, NC (US)

(21) Appl. No.: 19/492,517

(22) PCT Filed: Sep. 22, 2023
(86) PCT No.: PCT/US2023/033483

§ 371 Date: Dec. 11, 2025

Related U.S. Application Data
(60) Provisional application No. 63/376,817, filed on Sep. 23, 2022.

(51) Int. Cl. A61B 17/3213; A61B 17/00; A61B 17/3211

(52) U.S. Cl. CPC A61B 17/3213; A61B 2017/00398; A61B 2017/00734; A61B 2017/32113

(57) ABSTRACT

A surgical knife for cutting tissue may include a handle assembly and a cartridge configured for removably coupling to the handle assembly. The handle assembly may include a housing, an electric motor disposed within the housing, a battery disposed within the housing and configured for powering the electric motor, a gear train disposed within the housing and coupled to the electric motor, and a shaft coupled to the gear train and extending outside of the housing. The shaft may be configured for oscillating relative to the housing upon activation of the electric motor. The cartridge may include a frame defining at least a portion of a track, a rail configured for oscillating along the track, a surgical blade removably coupled to the rail and configured for oscillating therewith, and a guard coupled the frame and configured for controlling a cutting depth of the surgical blade into the tissue.

### CROSS REFERENCE TO RELATED APPLICATIONS

This application claims the benefit of U.S. Provisional Application No. 63/376,817, filed on Sep. 23, 2022, which is incorporated herein by reference in its entirety.

### FIELD OF THE DISCLOSURE

The present disclosure relates generally to medical instruments and more particularly to surgical knives and related methods of using the same, for example, for surgical excision of tissue of a patient.

### BACKGROUND OF THE DISCLOSURE

Surgical excision of tissue of a patient may be necessary in various medical scenarios. For example, patients with a full-thickness burn injury often may require excision of devitalized tissue prior to application of skin grafts to heal the affected areas of injury. To perform such excisions, burn surgeons generally may use manual dermatomes, such as Goulian/Weck knives and Watson blades. However, these cutting tools often may be too large to fit into intricate and complicated spaces of the body (e.g., between fingers, between toes, within the curvature of the axillae, along the nose, behind the ears, etc.) when performing excision. The smallest of the currently-available manual dermatomes, the Goulian/Weck knife, gives the surgeon the ability to choose between 8/1000-inch, 10/1000-inch, and 12/1000-inch depths of excision. This often may lead to over-or under-excision in areas that require precision beyond that which the tool can provide. Precision also may be compromised by the pressure with which the surgeon applies the tool to the patient's skin and by the angle at which the surgeon pushes the blade against more awkward contours of the tissue being cut. Accordingly, there are multiple variables, including the size of the cutting tool, the guards used with the tool, and the dexterity of the surgeon, that can affect the depth at which tissue is excised, which potentially may cause harm in areas where precise excision is difficult to achieve.

A need therefore exists for improved devices and methods for surgical excision of tissue of a patient, particularly at intricate and complicated spaces of the body, which may overcome one of more of the challenges associated with existing cutting tools and methods of their use.

### SUMMARY OF THE DISCLOSURE

The present disclosure provides surgical knives and related methods of using the same, for example, for surgical excision of tissue of a patient.

In one aspect, a surgical knife for cutting tissue is provided. In some embodiments, the surgical knife may include a handle assembly and a cartridge configured for removably coupling to the handle assembly. The handle assembly may include a housing, an electric motor, a battery, a gear train, and a shaft. The housing may have a proximal end and a distal end disposed opposite one another along a longitudinal axis of the housing. The electric motor may be disposed within the housing. The battery may be disposed within the housing and configured for powering the electric motor. The gear train may be disposed within the housing and coupled to the electric motor. The shaft may be coupled to the gear train and extend outside of the housing, and the shaft may be configured for oscillating relative to the housing upon activation of the electric motor. The cartridge may include a frame, a rail, a surgical blade, and a guard.

The frame may define at least a portion of a track. The rail may be configured for oscillating along the track. The surgical blade may be removably coupled to the rail and configured for oscillating therewith. The guard may be coupled the frame and configured for controlling a cutting depth of the surgical blade into the tissue.

In some embodiments, the housing may be configured for being grasped by a user for cutting the tissue. In some embodiments, the housing may have a circular cross-sectional shape taken perpendicular to the longitudinal axis of the housing. In some embodiments, the housing may be formed of a metal. In some embodiments, the metal may include aluminum or stainless steel. In some embodiments, at least a portion of an exterior surface of the housing may include a textured surface. In some embodiments, the textured surface may include a knurled surface. In some embodiments, the motor, the battery, and the gear train may be enclosed within the housing. In some embodiments, the housing may include a cap disposed at the proximal end of the housing and removably coupled to a remainder of the housing. In some embodiments, the housing may be configured for allowing the battery to be removed from the housing when the cap is decoupled from the remainder of the housing. In some embodiments, the handle assembly may include one or more charging connectors disposed at the proximal end of the housing and configured for engaging one or more mating charging connectors of a charger for charging the battery while the battery is disposed within the housing.

In some embodiments, the electric motor may be fixedly mounted within the housing. In some embodiments, the electric motor may be a DC motor. In some embodiments, the electric motor may include a motor housing and an output shaft configured to rotate relative to the motor housing upon activation of the motor. In some embodiments, the output shaft may be centered on the longitudinal axis of the housing. In some embodiments, the gear train may be coupled to the output shaft. In some embodiments, the battery may be rechargeable. In some embodiments, the battery may be a lithium ion battery. In some embodiments, the handle assembly may include a rheostat in operable communication with the battery and the motor and configured for adjusting a speed of the motor and an oscillation speed of the shaft. In some embodiments, the handle assembly may include an off button or an off switch in operable communication with the battery and the motor and configured for deactivating the motor regardless of a setting of the rheostat.

In some embodiments, the gear train may include a pinion gear and a ring gear engaged with one another. In some embodiments, the pinion gear may be coupled to an output shaft of the electric motor. In some embodiments, the shaft may be coupled to the ring gear by a linkage. In some embodiments, the shaft may be movably coupled to the linkage at a pin joint. In some embodiments, the linkage may be movably coupled to a post extending from the ring gear and offset from a center of the ring gear. In some embodiments, the shaft may be centered on the longitudinal axis of the housing. In some embodiments, the shaft may extend beyond the distal end of the housing. In some embodiments, the shaft may include a head portion configured for engaging the rail when the cartridge is coupled to the handle assembly.

In some embodiments, the cartridge may be configured for removably coupling to the handle assembly via a twist-lock mechanism. In some embodiments, the twist-lock mechanism may include a first connector of the handle assembly and a second connector of the cartridge. In some embodiments, the frame may be configured for removably coupling to the handle assembly. In some embodiments, the track may have a linear shape. In some embodiments, the rail may have a linear shape. In some embodiments, the surgical blade may have a linear shape. In some embodiments, the surgical blade may be rigid. In some embodiments, the guard may have a linear shape. In some embodiments, the track may have a curved shape. In some embodiments, the rail may have a curved shape. In some embodiments, the rail may have a linear shape but may be flexible and configured for conforming to the curved shape of the track. In some embodiments, the surgical blade may have a curved shape. In some embodiments, the surgical blade may have a linear shape but may be flexible and configured for conforming to the curved shape of the track. In some embodiments, the guard may have a curved shape. In some embodiments, the cartridge may include one or more rollers, one or more bearings, or one or more lubricants configured for facilitating oscillation of the rail and the surgical blade along the track. In some embodiments, the rail may include a plurality of posts each configured for engaging a respective hole defined in the surgical blade when the surgical blade is coupled to the rail, and the guard may include a plurality slots each configured for receiving one of the posts therein. In some embodiments, the guard may include an opening configured for allowing tissue cut by the surgical blade to pass therethrough. In some embodiments, the surgical blade may include a cutting edge, and the guard may be configured for covering a portion of the surgical blade while the cutting edge extends beyond the guard, thereby controlling the cutting depth of the surgical blade into the tissue. In some embodiments, the guard may be movably coupled to the frame. In some embodiments, the guard may be configured for moving relative to the frame between a plurality of positions each associated with a different cutting depth of the surgical blade into the tissue. In some embodiments, the cartridge may include a locking mechanism configured for selectively locking the guard relative to the frame in each of the plurality of positions. In some embodiments, the guard may be configured for moving relative to the frame between an open position, in which the surgical blade may be removed from the rail, and a closed position, in which the surgical blade is retained on the rail between the frame and the guard.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1A is a side partial cross-sectional view of an example surgical knife in accordance with embodiments of the disclosure, showing a handle assembly and a first cartridge of the surgical knife coupled to one another.

FIG. 1B is a side view of a portion of a housing of the handle assembly of the surgical knife of FIG. 1A.

FIG. 1C is a detailed side view of an interface between the handle assembly and the first cartridge of the surgical knife of FIG. 1A.

FIG. 1D is an end partial cross-sectional view of a portion of the handle assembly of the surgical knife of FIG. 1A.

FIG. 1E is a detailed side view of the first cartridge of the surgical knife of FIG. 1A, showing a frame, a rail, a surgical blade, and a guard of the first cartridge, with the guard in an open configuration relative to the frame.

FIG. 1F is a detailed side view of a second cartridge of the surgical knife of FIG. 1A, showing a frame, a rail, a surgical blade, and a guard of the second cartridge, with the guard in an open configuration relative to the frame.

FIG. 1G is a detailed side view of a third cartridge of the surgical knife of FIG. 1A, showing a frame, a rail, a surgical blade, and a guard of the third cartridge, with the guard in an open configuration relative to the frame.

FIG. 1H is a detailed top view of the third cartridge of the surgical knife of FIG. 1A, showing the guard in a closed configuration relative to the frame.

FIG. 1I is a detailed end view of the third cartridge of the surgical knife of FIG. 1A, showing the guard in the closed configuration relative to the frame.

FIG. 1J is a detailed side view of the third cartridge of the surgical knife of FIG. 1A, showing the guard in the closed configuration relative to the frame.

FIG. 1K is a detailed end view of the third cartridge of the surgical knife of FIG. 1A, showing the guard in the closed configuration relative to the frame.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

### Overview

The present disclosure provides surgical knives and related methods of using the same, for example, for surgical excision of tissue of a patient. As described herein, a surgical knife may be an electric-handled device that comes with a myriad of attachable cartridge-style blades that will oscillate at variable speeds, as may be controlled by a rheostat dial of the handle. The cartridges themselves may be provided in various head sizes, bevels, and concave/convex curvatures, so as to more easily access and excise difficult to reach areas or body regions that are heavily contoured and not as conducive to conventional excision techniques-while maintaining or improving specificity in tissue excision and precision in the depth of tissue being excised.

The disclosed surgical knife may be used primarily for excision and debridement of devitalized tissue in surgeries requiring tangential excision of the skin. The surgical knife may have a stainless steel or aluminum housing with removable cartridges. The housing may contain a removable lithium ion battery, a motor, a gear train, and a shaft arranged in a manner to cause a surgical razor blade (located in the removable cartridge) to oscillate to remove dead tissue. The housing may have a screw on cap for access to the battery so that the battery may be quickly replaced during an OR case. The cartridge may include a stainless steel frame and a rail that slides along a track of the frame and carries the surgical blade during oscillation. The cartridge also may include a guard that controls the depth of tissue being excised by the surgical blade.

The guard may flip open to allow the blade to be easily and safely exchanged during the OR case. Various different cartridges may be provided-each differing in head size, blade angle/bevel, and convexity/concavity of the cartridge itself-to best achieve accurate excision in difficult-to-manage areas (e.g., between the fingers or the toes). Each cartridge may include an adjustable guard that allows the surgeon to select a desired excision depth, such as within a range between 0/1000-inch and 20/1000-inch. Notably, the adjustable guard may be adjusted to a specific depth at any time during an OR procedure. The electric motor may control the oscillating surgical blade for easy tissue removal in tight spaces that would otherwise prove difficult to navigate with manual back-and-forth movement of the knives currently available on the market. The disclosed surgical knife may be used in various different applications, including, but not limited to, tissue removal, eschar removal, skin harvesting, and debridement of foreign bodies.

### Example Surgical Knives and Methods

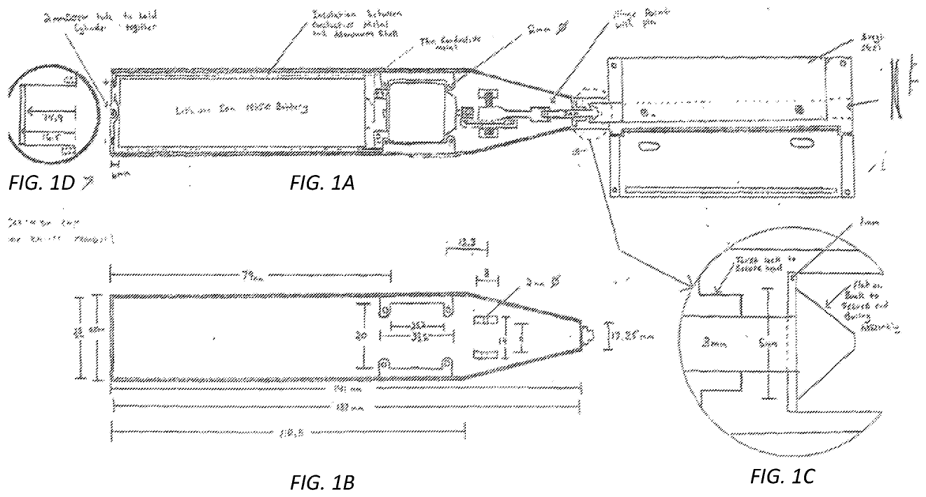

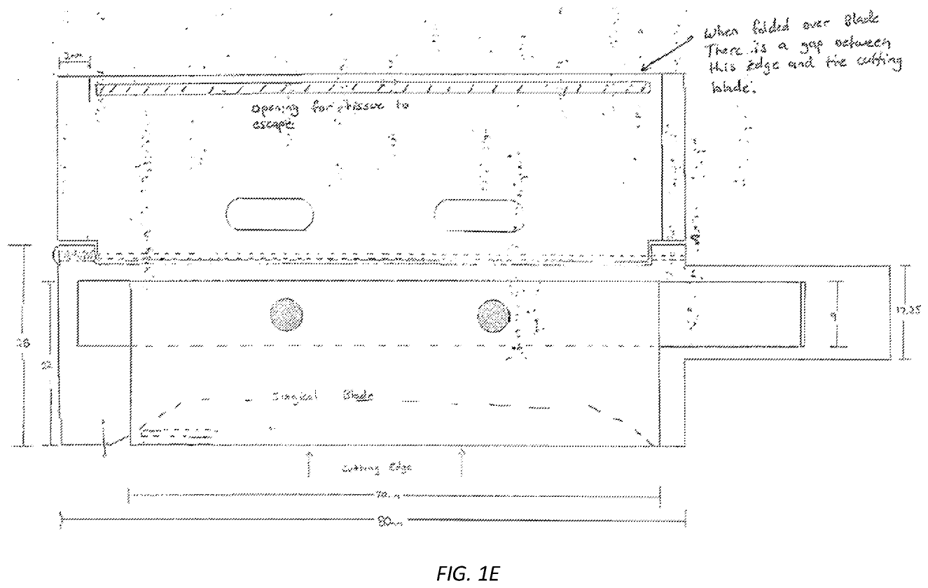

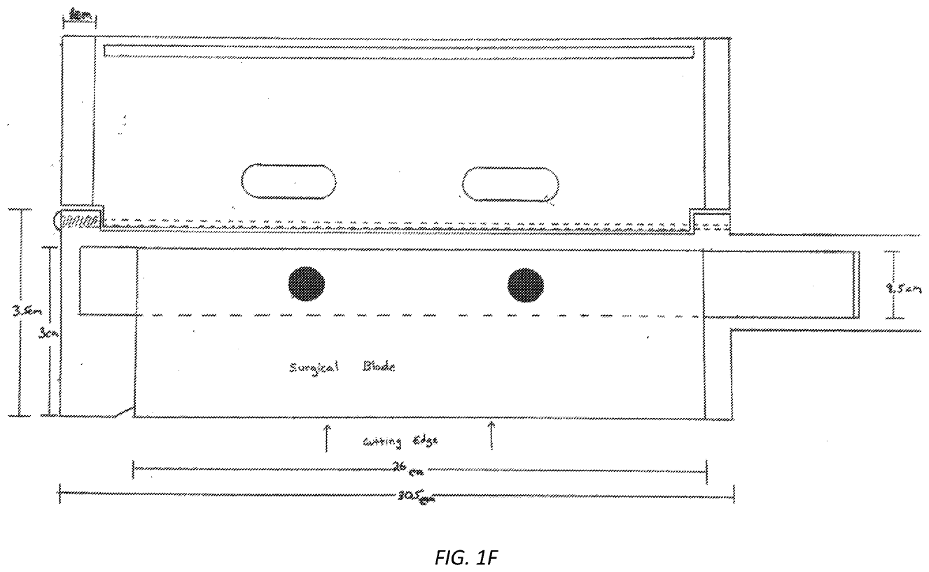

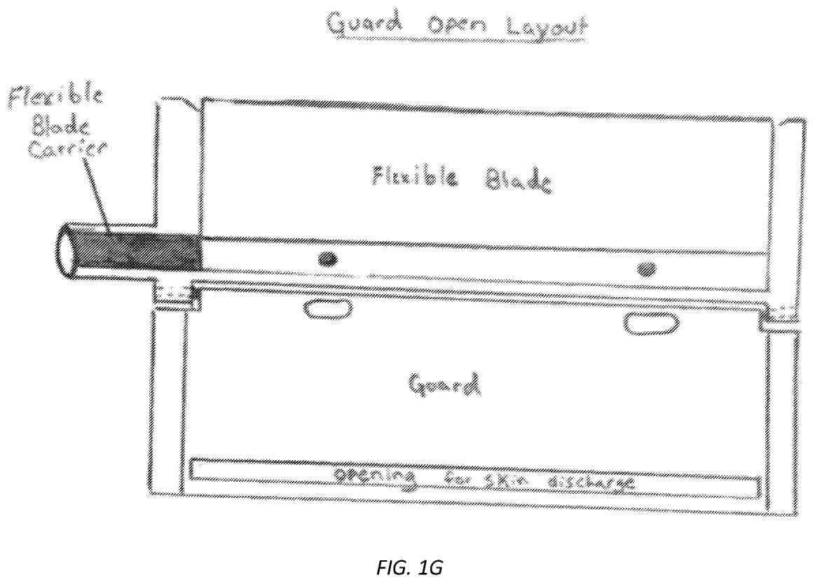

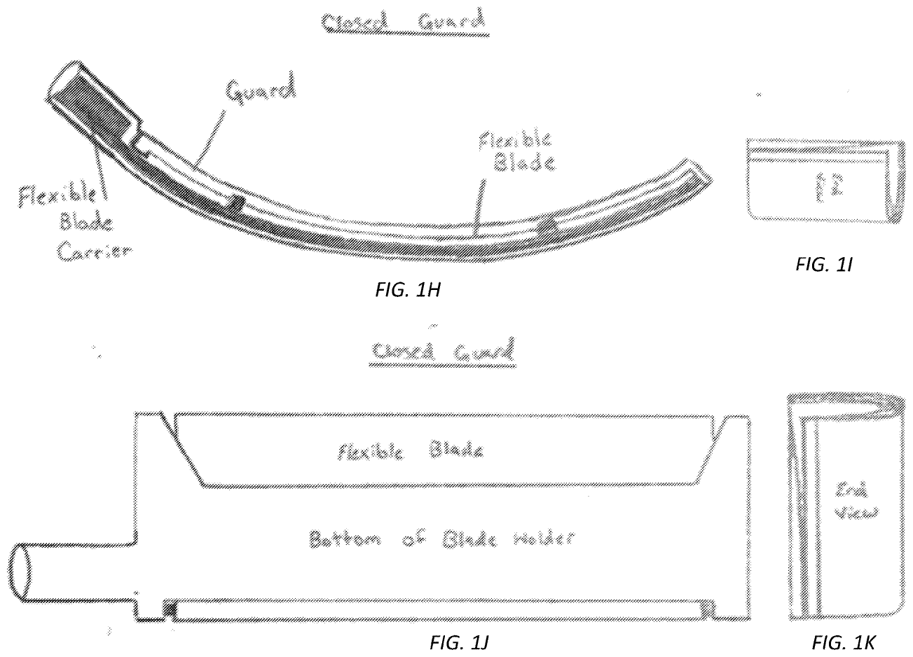

FIGS. 1A-1K illustrate an example surgical knife (which also may be referred to as an “electric surgical knife” or a “surgical excision tool”) and components thereof in accordance with embodiments of the disclosure. As shown in FIG. 1A, the surgical knife may include a handle assembly and a first cartridge configured for removably coupling to the handle assembly. As discussed above, the surgical knife may include a plurality of different cartridges each configured for removably coupling to the handle assembly, with the various cartridges having different sizes, shapes, and/or configurations suited for different uses of the surgical knife. In this manner, the handle assembly and the plurality cartridges may be provided as a kit, allowing a user to select and switch between different cartridges, as desired. FIG. 1F illustrates an example second cartridge having larger dimensions than the first cartridge and generally sized in a manner similar to a Watson blade. FIGS. 1G-1K illustrate an example third cartridge having a curved configuration to provide ease of use for excising tissue that is challenging to access with a linear configuration. It will be appreciated that the different cartridges shown in FIGS. 1A and 1E-1K are merely examples, and that still other sizes, shapes, and configurations of cartridges may be provided for use with the handle assembly and included in a kit for the surgical knife.

In FIG. 1A, the first cartridge is shown coupled to the handle assembly. As shown, the handle assembly may include a housing, an electric motor, a battery, a gear train, and a shaft. The housing may have an elongate shape, with a proximal end and a distal end disposed opposite one another along a longitudinal axis of the housing. As shown in FIG. 1A, the first cartridge may be coupled to the handle assembly at the distal end of the handle assembly. The second cartridge, the third cartridge, and additional cartridges may be configured for coupling to the handle assembly in a similar manner. The electric motor may be disposed within the housing.

The battery may be disposed within the housing and configured for powering the electric motor. The gear train may be disposed within the housing and coupled to the electric motor. The shaft may be coupled to the gear train and extend outside of the housing, and the shaft may be configured for oscillating relative to the housing upon activation of the electric motor. As shown in FIGS. 1A and 1E, the first cartridge may include a frame, a rail, a surgical blade, and a guard. The frame may define at least a portion of a track, and the rail may be configured for oscillating along the track. The surgical blade may be removably coupled to the rail and configured for oscillating therewith. The guard may be coupled the frame and configured for controlling a cutting depth of the surgical blade into the tissue. As shown in FIGS. 1F-1K, the second cartridge and the third cartridge each may include similar components generally configured in a similar manner.

In some embodiments, the housing may be configured for being grasped by a user for cutting the tissue. In some embodiments, as shown, the housing may have a circular cross-sectional shape taken perpendicular to the longitudinal axis of the housing. In some embodiments, the housing may be formed of a metal, such as aluminum or stainless steel. In some embodiments, at least a portion of an exterior surface of the housing may include a textured surface to facilitate gripping of the handle assembly by a user. As an example, the textured surface may include a knurled surface. In some embodiments, as shown, the motor, the battery, and the gear train may be enclosed within the housing. In some embodiments, the housing may include a cap disposed at the proximal end of the housing and removably coupled to a remainder of the housing. In this manner, the housing may be configured for allowing the battery to be removed from the housing when the cap is decoupled from the remainder of the housing. In some embodiments, as shown, the handle assembly may include one or more charging connectors disposed at the proximal end of the housing and configured for engaging one or more mating charging connectors of a charger for charging the battery while the battery is disposed within the housing.

As shown, the electric motor may be fixedly mounted within the housing. In some embodiments, the electric motor may be a DC motor. In some embodiments, as shown, the electric motor may include a motor housing and an output shaft configured to rotate relative to the motor housing upon activation of the motor. In some embodiments, as shown, the output shaft may be centered on the longitudinal axis of the housing. In some embodiments, as shown, the gear train may be coupled to the output shaft. In some embodiments, the battery may be rechargeable. In some embodiments, the battery may be a lithium ion battery. In some embodiments, the handle assembly may include a rheostat in operable communication with the battery and the motor and configured for adjusting a speed of the motor and an oscillation speed of the shaft. In some embodiments, the handle assembly may include an off button or an off switch in operable communication with the battery and the motor and configured for deactivating the motor regardless of a setting of the rheostat. In this manner, the user may quickly halt oscillation of the surgical blade, when desired, by pressing the off button or moving the off switch.

As shown, the gear train may include a pinion gear and a ring gear engaged with one another. In some embodiments, as shown, the pinion gear may be coupled to the output shaft of the electric motor. In some embodiments, as shown, the shaft may be coupled to the ring gear by a linkage. In some embodiments, as shown, the shaft may be movably coupled to the linkage at a pin joint. In some embodiments, as shown, the linkage may be movably coupled to a post extending from the ring gear and offset from a center of the ring gear. In some embodiments, as shown, the shaft may be centered on the longitudinal axis of the housing. In some embodiments, as shown, the shaft may extend beyond the distal end of the housing. In some embodiments, as best shown in FIG. 1C, the shaft may include a head portion configured for engaging the rail when one of the cartridges is coupled to the handle assembly.

In some embodiments, as shown, each of the cartridges may be configured for removably coupling to the handle assembly via a twist-lock mechanism. In some embodiments, the twist-lock mechanism may include a first connector of the handle assembly and a second connector of the respective cartridge. In some embodiments, as shown, the frame may be configured for removably coupling to the handle assembly. In some embodiments, the track may have a linear shape, the rail may have a linear shape, the surgical blade may have a linear shape, and the guard may have a linear shape, as shown for each of the first cartridge and the second cartridge. In some embodiments, the surgical blade may be rigid. In some embodiments, the track may have a curved shape, as shown for the third cartridge. In some such embodiments, the rail may have a curved shape. In other embodiments, the rail may have a linear shape but may be flexible and configured for conforming to the curved shape of the track. In some embodiments, the surgical blade may have a curved shape. In other embodiments, the surgical blade may have a linear shape but may be flexible and configured for conforming to the curved shape of the track. In some embodiments, the guard may have a curved shape, as shown for the third cartridge.

In some embodiments, each of the cartridges may include one or more rollers, one or more bearings, or one or more lubricants configured for facilitating oscillation of the rail and the surgical blade along the track. In some embodiments, as shown, the rail may include a plurality of posts each configured for engaging a respective hole defined in the surgical blade when the surgical blade is coupled to the rail, and the guard may include a plurality of slots each configured for receiving one of the posts therein. In some embodiments, as shown, the guard may include an opening configured for allowing tissue cut by the surgical blade to pass therethrough. In some embodiments, the surgical blade may include a cutting edge, and the guard may be configured for covering a portion of the surgical blade while the cutting edge extends beyond the guard, thereby controlling the cutting depth of the surgical blade into the tissue. In some embodiments, the guard may be movably coupled to the frame. In some embodiments, the guard may be configured for moving relative to the frame between a plurality of positions each associated with a different cutting depth of the surgical blade into the tissue. In some embodiments, each of the cartridges may include a locking mechanism configured for selectively locking the guard relative to the frame in each of the plurality of positions. In some embodiments, the guard may be configured for moving relative to the frame between an open position, as shown in FIGS. 1A, 1E, 1F, and 1G, in which the surgical blade may be removed from the rail, and a closed position, as shown in FIGS. 1H-1K, in which the surgical blade is retained on the rail between the frame and the guard.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment. The term “based at least in part on” and “based on” are synonymous terms which may be used interchangeably herein.

1. A surgical knife for cutting tissue, the surgical knife comprising:
a handle assembly comprising:
a housing having a proximal end and a distal end disposed opposite one another along a longitudinal axis of the housing;
an electric motor disposed within the housing;
a battery disposed within the housing and configured for powering the electric motor;
a gear train disposed within the housing and coupled to the electric motor; and
a shaft coupled to the gear train and extending outside of the housing, the shaft configured for oscillating relative to the housing upon activation of the electric motor; and
a cartridge configured for removably coupling to the handle assembly, the cartridge comprising:
a frame defining at least a portion of a track;
a rail configured for oscillating along the track;
a surgical blade removably coupled to the rail and configured for oscillating therewith; and
a guard coupled the frame and configured for controlling a cutting depth of the surgical blade into the tissue.

2. (canceled)

3. The surgical knife of claim 1, wherein the housing has a circular cross-sectional shape taken perpendicular to the longitudinal axis of the housing.

4-5. (canceled)

6. The surgical knife of claim 1, wherein at least a portion of an exterior surface of the housing comprises a textured surface.

7. (canceled)

8. The surgical knife of claim 1, wherein the motor, the battery, and the gear train are enclosed within the housing.

9. The surgical knife of claim 1, wherein the housing comprises a cap disposed at the proximal end of the housing and removably coupled to a remainder of the housing.

10. (canceled)

11. The surgical knife of claim 1, wherein the handle assembly further comprises one or more charging connectors disposed at the proximal end of the housing and configured for engaging one or more mating charging connectors of a charger for charging the battery while the battery is disposed within the housing.

12. The surgical knife of claim 1, wherein the electric motor is fixedly mounted within the housing.

13. (canceled)

14. The surgical knife of claim 1, wherein the electric motor comprises a motor housing and an output shaft configured to rotate relative to the motor housing upon activation of the motor.

15. (canceled)

16. The surgical knife of claim 14, wherein the gear train is coupled to the output shaft.

17-18. (canceled)

19. The surgical knife of claim 1, wherein the handle assembly further comprises a rheostat in operable communication with the battery and the motor and configured for adjusting a speed of the motor and an oscillation speed of the shaft.

20. (canceled)

21. The surgical knife of claim 1, wherein the gear train comprises a pinion gear and a ring gear engaged with one another.

22. The surgical knife of claim 21, wherein the pinion gear is coupled to an output shaft of the electric motor; wherein the shaft is coupled to the ring gear by a linkage; and/or wherein the shaft is movably coupled to the linkage at a pin joint.

23-24. (canceled)

25. The surgical knife of claim 22, wherein the linkage is movably coupled to a post extending from the ring gear and offset from a center of the ring gear.

26. The surgical knife of claim 1, wherein the shaft is centered on the longitudinal axis of the housing.

27. The surgical knife of claim 1, wherein the shaft extends beyond the distal end of the housing.

28. The surgical knife of claim 1, wherein the shaft comprises a head portion configured for engaging the rail when the cartridge is coupled to the handle assembly.

29-31. (canceled)

32. The surgical knife of claim 1, wherein the track, rail, surgical blade, and/or guard has a linear or curved shape.

33-42. (canceled)

43. The surgical knife of claim 1, wherein the cartridge further comprises one or more rollers, one or more bearings, or one or more lubricants configured for facilitating oscillation of the rail and the surgical blade along the track.

44. The surgical knife of claim 1, wherein the rail comprises a plurality of posts each configured for engaging a respective hole defined in the surgical blade when the surgical blade is coupled to the rail, and wherein the guard comprises a plurality slots each configured for receiving one of the posts therein.

45. (canceled) Page

46. The surgical knife of claim 1, wherein the surgical blade comprises a cutting edge, and wherein the guard is configured for covering a portion of the surgical blade while the cutting edge extends beyond the guard, thereby controlling the cutting depth of the surgical blade into the tissue.

47. (canceled)

48. The surgical knife of claim 1, wherein the guard is configured for moving relative to the frame between a plurality of positions each associated with a different cutting depth of the surgical blade into the tissue.

49. (canceled)

50. The surgical knife of claim 1, wherein the guard is configured for moving relative to the frame between an open position, in which the surgical blade may be removed from the rail, and a closed position, in which the surgical blade is retained on the rail between the frame and the guard.
